(19) United States
(12) Patent Application Publication
(10) Pub. No.: US 2010/0267733 A1
(43) Pub. Date: Oct. 21, 2010

(54) Synergistic Modulation of Microglial Activation by Nicotine and THC

(75) Inventors: Roland D. Shytle, Largo, FL (US); Jun Tan, Tampa, FL (US); Jared Ehrhart, Tampa, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 12/772,732

(22) Filed: May 3, 2010

Related U.S. Application Data
(63) Continuation of application No. PCT/US2008/082208, filed on Nov. 3, 2008.
(60) Provisional application No. 60/984,999, filed on Nov. 2, 2007.

(51) Int. Cl. A61K 31/352; A61K 31/465; A61K 31/4439; A61K 31/505; A61K 31/4965; A61K 31/4985; A61P 29/00; A61P 25/00; A61P 25/16; A61P 25/28

(52) U.S. Cl. 514/250; 514/343; 514/339; 514/454; 514/274; 514/255.03

(57) ABSTRACT

Treatment of microglial cells with nicotine and THC synergistically attenuate the microglial activation. Using microglial activation, the combination of THC and nicotine interact synergistically reduced LPS induced TNF-α release, showing that the combination of THC and nicotine clinically have greater efficacy in reducing neuroinflammation with less side effects than either drug given alone. CD40 signaling was found critically involved in pathological activation of microglial cells. This invention is also relevant to peripheral inflammation as well thru macrophages. In addition, other cannabinoids and other nicotinic-like medications currently in development are also covered under this discovery.

### CROSS REFERENCE TO RELATED APPLICATIONS

This application is a continuation of prior filed International Application, Serial Number PCT/US2008/082208 filed Nov. 3, 2008, which claims priority to U.S. provisional patent application No. 60/984,999 filed Nov. 2, 2007 which is hereby incorporated by reference into this disclosure.

### STATEMENT OF GOVERNMENT SUPPORT

This invention was made with Government support under Grant No. R21 AG031037 awarded by the National Institutes of Health. The Government has certain rights in the invention.

### FIELD OF INVENTION

This invention relates to a method for reducing cytokine release in the brain. Specifically, the invention involves using nicotine and THC to reduce inflammatory response and neurodegenerative disease.

### BACKGROUND OF THE INVENTION

It is currently believed that resident immune cells in the central nervous system (CNS) play a critical role in the etiology of various neurodegenerative diseases. Chronic activation of microglia is believed to trigger and maintain an inflammatory response, which may ultimately lead to neuronal cell death, such as that observed in Alzheimer's disease, HIV dementia, Parkinson's disease, prion disease, amyotrophic lateral sclerosis, and multiple sclerosis (Eikelenboom, P., et al., Neuroinflammation in Alzheimer's disease and prion disease. Glia, 2002, 40:232-239; Garden, G. A. Microglia in human immunodeficiency virus-associated neurodegeneration. Glia, 2002, 40:240-251; Tan, J., et al., Activation of microglial cells by the CD40 pathway: relevance to multiple sclerosis. J. Neuroimmunol., 1999, 97:77-85). Societal costs of these diseases are profound. For example, Alzheimer's disease currently affects an estimated 4.5 million Americans, costing the U.S. more than $100 billion annually. Finding a treatment that could delay onset by five years could reduce the number of individuals with AD by nearly 50 percent after 50 years.

Inflammatory mechanisms play a significant role in the pathogenesis of Alzheimer's disease (AD) and other common neurodegenerative disorders. Particularly in the case of AD, several lines of evidence have been proposed to indicate that inflammation is a central component of the disease process. Neuropathological findings place microglial cells and astrocytes in close association with senile plaques and/or neurofibrillary tangles of AD.

Microglia constitute a widely distributed network of immunoprotective cells in the brain. Cells activated by lipopolysaccharide (LPS), a bacterial endotoxin, release neurotoxic cytokines, such as Tumor Necrosis Factor (TNF-α), which result in neuronal death. Chronic activation of immune cells exposes the CNS to elevated levels of potentially neurotoxic molecules, including pro-inflammatory cytokines, complement proteins, proteinases, and reactive oxygen species (ROS) (McGuire, S. O., et al., Tumor necrosis factor alpha is toxic to embryonic mesenchalic dopamine neurons. Exp. Neurol., 2001, 169:219-230; Chao, C. C., et al., Interleukin-1 and tumor necrosis factor-α synergistically mediate neurotoxicity: involvement of nitric oxide and of N-methyl-D-aspartate receptors. Brain Behav. Immun., 1995, 9:355-365). Alternatively, dysregulation of microglial activation may prevent appropriate immune responses necessary to respond to neural insults (Streit, W. J. Microglia and neuroprotection: implications for Alzheimer's disease. Brain Res. Rev., 2005, 48:234-9).

CD40 ligation is an essential stimulatory signal to microglial activation. CD40 and its ligand are key immunoregulatory molecules that provide co-stimulatory input to cells of the innate and adaptive immune system (Alderson, M. R., et al., CD40 expression by human monocytes: regulation by cytokines and activation of monocytes by the ligand for CD40. J. Exp. Med., 1993, 178:669-74; van Kooten, C., Banchereau, J., CD40-CD40 ligand. J. Leukoc. Biol., 2000, 67:2-17; Grewal, I. S., Flavell, R. A. CD40 and CD154 in cell-mediated immunity. Annu. Rev. Immunol., 1998, 16:111-135) The classic stimulatory signal for microglial activation is propagated by T-cell release of interferon-gamma (IFN-γ), which sensitizes microglia by upregulating the expression of immunoregularoty molecues, including CD40, on the cell surfaces (Boehm, et al., Cellular responses to interferon-gamma. Annu. Rev. Immunol., 1997, 15:749-795; Seder, R. A., Paul, W. E. Acquisition of lymphokine-producing phenotype by CD4⁺ T cells. Annu. Rev. Immunol., 1994, 12:635-673). Further, activation of the Janus kinase/signal transducer and activation of transcription (JAK/STAT) signaling pathway plays an essential role in this IFN induced microglial CD40 expression (Nguyen, V. T., Benveniste, E. N. Involvement of STAT-1 and ets family members in interferon-gamma induction of CD40 transcription in microglia/macrophages. J. Biol. Chem., 2000, 275:23674-84; Leonard, W. J., O'Shea, J. J. Jaks and STATs: biological implications. Annu. Rev. Immunol., 1998, 16:293-322).

Thus, novel therapies which can reduce microglial activation are useful in treating a variety of neurodegenerative disorders, such as Alzheimer's disease, Parkinson's disease, ALS, and HIV related dementia.

### SUMMARY OF THE INVENTION

The role of acetylcholine (ACh) has been investigated in microglial activation induced by bacterial endotoxin, lypopolysaccharide (LPS). ACh and nicotine pretreatment inhibited LPS-induced TNF-α release in murine derived microglial cells, an effect prevented by nonselective nicotinic antagonist, mecamylamine, and by α7 selective nicotinic antagonist, α-bungarotoxin. This indicates a cholinergic pathway is utilized to regulate microglial activation through α7 nicotinic receptor subtype.

Nicotine, the active ingredient in tobacco, and THC, the active ingredient in marijuana, both possess immune suppressing properties. No studies have investigated the neuroimmunological effects of chronic combined nicotine and THC exposure in normal animals or animal models of neurodegenerative diseases. A combination treatment of nicotine/THC was tested as a therapeutic of neurodegenerative disorders, like AD, along with the signaling mechanisms of the innate immune system and APP processing after treatment.

Disclosed is a method of modulating inflammatory response in a by administering at least one cannabinoid and at least one nicotinic compound. In some embodiments, the cannabinoid is a cannabinoid-2 receptor agonist, which include, without limitation, delta-9-tetrahydrocannabinol, cannabidiol, dronabinol, JHW 015, anandamide, 2-arachidonyl glyceride, 2-arachidonyl glyceryl ether, O-arachidonoyl-ethanolamine, nabilone, PRS-211,092, CP 55,940 WIN-55212-2, JWH 133, SR 144528, and levonantradol. The nicotinic compound is which include, without limitation, nicotine, epibatidine, acetylcholine, cytosine, carbachol, dimethlphenylpiperazimium, and varenicline. The composition modulates microglia-activated Th1 and Th2 immune responses. In specific embodiments, the inflammatory response is induced by LPS.

In some embodiments, the composition is administered intrathecally, subcutaneously or intravenously, and specific embodiments provide that the composition is administered within the range of 0.3 and 3 mg/kg/day.

Also disclosed is a method of treating a neurodegenerative disease in a patient by administering at least one cannabinoid and at least one nicotinic compound. In some embodiments, the cannabinoid is a cannabinoid-2 receptor agonist, which include, without limitation, delta-9-tetrahydrocannabinol, cannabidiol, dronabinol, JHW 015, anandamide, 2-arachidonyl glyceride, 2-arachidonyl glyceryl ether, O-arachidonoyl-ethanolamine, nabilone, PRS-211,092, CP 55,940 WIN-55212-2, JWH 133, SR 144528, and levonantradol. The nicotinic compound is which include, without limitation, nicotine, epibatidine, acetylcholine, cytosine, carbachol, dimethlphenylpiperazimium, and varenicline. The composition modulates microglia-activated Th1 and Th2 immune responses. The method is useful in treating neurodegenerative diseases, including Parkinson's disease, Alzheimer's disease, multiple sclerosis, Tay Sach's disease, Rett Syndrome, lysosomal storage diseases, HIV dementia, prion disease, ischemia, ataxia, and amyotrophic lateral sclerosis. However, the method is effective at treating other neurodegenerative diseases as well, which are envisioned by this invention. In specific embodiments of the invention, the method is used to treat amyotrophic lateral sclerosis.

In some embodiments, the composition is administered systemically intrathecally, and in specific embodiments, the composition is administered subcutaneously or intravenously. The nicotinic compound is administered at 0.2 mg/kg/day and the cannabinoid is administered within the range of 0.3 and 3 mg/kg/day.

A composition is also disclosed, comprising at least one cannabinoid and at least one nicotinic compound. In certain embodiments, the cannabinoid is a cannabinoid-2 receptor agonist, such as delta-9-tetrahydrocannabinol, cannabidiol, dronabinol, JHW 015, anandamide, 2-arachidonyl glyceride, 2-arachidonyl glyceryl ether, O-arachidonoyl-ethanolamine, nabilone, PRS-211,092, CP 55,940 WIN-55212-2, JWH 133, SR 144528, and levonantradol. The cannabinoid compound may be administered within the range of 0.3 and 3 mg/kg/day. In some embodiments, the nicotinic compound used includes nicotine, epibatidine, acetylcholine, cytosine, carbachol, dimethlphenylpiperazimium, and varenicline. The nicotinic compound may be administered at 0.2 mg/kg/day.

The effects of a cannabinoid agonist were investigated on CD40 expression and its function by cultured microglial cells activated by LPS, IFN-γ, Aβ, and CD-40L.

### BRIEF DESCRIPTION OF THE DRAWINGS

For a fuller understanding of the invention, reference should be made to the following detailed description, taken in connection with the accompanying drawings, in which:

FIG. 1 is a block graph showing the effect of nicotine on microglial activation induced by combined Aβ and IFN-γ peptide challenge, cultured microglial cells were pre-incubated with 10 pM nicotine (NIC) for 30 minutes and challenged with combined Aβ (1 pM) and IFN-y (100 ng/mL) for 12 hours. Microglial cell release of cytokines was measured for (A) TNF-α, (B) IL-6, and (C) IL-β.

FIG. 2 shows nicotine effects on microglial phagocytosis of Aβ₁₋₄₂ peptide. Primary cultured microglial cells (1×10⁵ cells/well in 24-well tissue-culture plates) were treated with FITC-tagged Aβ₁₋₄₂ (300 nM, pre-aggregated for 24 h at 37° C. in complete medium for 60 mm in the absence (control) or presence of nicotine (1, 5, or 10 pM). In addition, in parallel 24-well tissue-culture plates, microglial cells were incubated at 40° C. with the same treatment above. Cell supernatants and lysates were analyzed for (A) extracellular and (B) cell associated FITC-Aβ using a fluorometer.

FIG. 3 is a block graph showing cannabinoid agonist effects on microglial (N9) activation. Cultured microglial cells were incubated with 100 ng/mL LPS (LPS), a combination of IFN-γ (100 U/mL) and CD-40L (2 μg/mL), or a combination of Aβ₁₋₄₂ (1 μM) and CD-40L (2 μg/mL) and co-treated with JWH-015 (5 μM). Anti-CB₂ siRNA was added to cells, abolishing the ability of JWH-015 to reduce LPS-induced cytokine release (n=3, **p<0.05). ELISA analysis of microglial cell release of (A) TNF-α and (B) NO was measured and mean levels per mg of total protein shown. ANOVA and post hoc tests revealed significant differences between IFN-γ/CD-40L versus IFN-γ/CD-40L/JWH-015 (**p<0.05) and Aβ₁₋₄₂/CD-40L versus Aβ₁₋₄₂/CD-40L/JWH-015 (**p<0.001).

FIG. 4 is a graph showing CB2 stimulation modulates microglial phagocytic function. Mouse primary microglial cells were seeded at 5×10⁵ cells/well and treated with 3 μM Cy₃™-Aβ₁₋₄₂ with CD-40L protein (2.5 μg/mL), JWH-015 (5 μM) or both JWH-015 and CD-40L. Aβ mean band densities are represented as ratios to β-actin+/−SD (n=3 for each condition). ANOVA revealed significant differences between groups (JWH-015/Aβ versus CD-40L/Aβ and Aβ/CD-40L and Aβ/CD-40L versus JWH-015/CD-40L/Aβ; **p<0.005), and post hoc testing showed significant differences between CD-40L/Aβ and JWH-015/CD-40L/Aβ (**p<0.005).

FIG. 5 depicts the effects of Nicotine and THC alone and in combination on LPS-induced TNF-a release in microglia. Cultured microglial cells were plated in 24-well tissue-culture plates (Costar, Cambridge, Mass.), using minimum essential media supplemented with 5% fetal bovine serum, at 1×10 cells per well. The cells were pretreated for 30 mm with serial dilutions of either THC (10 uM-0.625 uM) and Nicotine (10 μM-0.625 μM). After pretreatment the cells were stimulated with LPS (100 ng/mL) for 4 hrs. Cell-free supernatants were collected and stored at −70° C. until analysis. The TNF-α level in the supernatants were examined using ELISA kits (R&D Systems) in strict accordance with the manufacturers' protocols. Cell lysates were also prepared and the Bio-Rad protein assay (Hercules, Calif.) was performed to measure total cellular protein. Results are shown as mean pg of TNF-α per mg of total cellular protein (+1-SEM).

FIG. 6 is a graph showing cultured microglial cells plated in a 24 well plate using the methods described above. The cells were then pre-treated for 30 min with serial dilutions of either THC (10 μM-0.625 μM) or nicotine (10 μM-0.625 μM). After pre-treatment the cells were stimulated with LPS (100 ng/mL) for 4 hours. The IL-6 levels in the supernatant were examined using ELISA kits in strict accordance with the manufacturer's protocols. Results are shown as mean pg of IL-6 per mg of total cellular protein (+/−SEM).

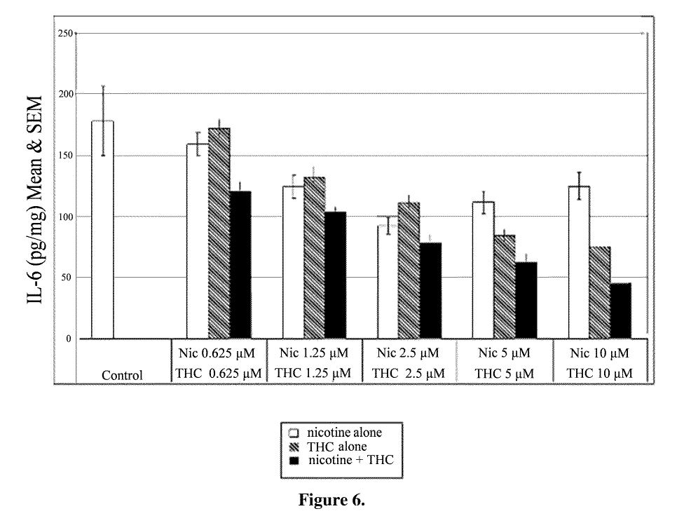

FIG. 7 is a graph depicting the combination of nicotine and THC reducing TNF-alpha expression stimulated by lipopolysaccharide (LPS), measured my ELISA. C57BL/6 mice were injected once intraperitonally (i.p.) with various concentrations of nicotine and THC, both individually and in combination. LPS was i.p. injected at the same time of drug administration. Mice were sacrificed 6 hrs. post injection, and tissues collected for analysis. Statistical analysis showed significance (P<0.0001) between the combination and its respective individual doses.

FIG. 8 is a graph depicting the combination of nicotine and THC reducing TNF-alpha expression in Swedish APP/PS1 (PSAPP) double transgenic Alzheimer mice measured by ELISA. Mice used were split into two age groups, old mice which were 16+ months of age and mid-aged mice were 11-12 months old. PSAPP mice were i.p. injected once daily for two weeks with various concentrations of nicotine and THC, administered alone and in combination. After the two week injection period, mice were sacrificed and tissues were collected for analysis. Statistical analysis showed significance (P<0.02) between old animals receiving a combination dose (THC 0.3 mg/kg & nicotine 0.3 mg/kg) and the old animals controls.

### DETAILED DESCRIPTION OF THE PREFERRED EMBODIMENT

“Patient” is used to describe an animal, preferably a human, to whom treatment is administered, including prophylactic treatment with the compositions of the present invention.

As used herein a cannabinoid receptors refers to cannabinoid receptor 2 (CB2), unless directed otherwise. The cannabinoid receptor is located in the membrane and on the surface of both brain and lymphoid cells and interacts with several endogenous natural ligands termed endo-cannabinoids and synthetic ligands. CB2 belongs to the family of G protein-coupled receptors characterized by seven trans-membrane loops interacting with the ligand on the outer surface of the cell and contain an intracellular signaling domain.

A “cannabinoid” as used herein is an “analog” of Δ⁹-tetrahydrocannabinol (THC) that retains the chemical structures of THC necessary for functional activity of THC and also contains certain chemical structures which differ from THC. The cannabinoid is a ligand of the cannabinoid receptor 2. The analog may be naturally occurring or synthetic. An example of a synthetic analog of a naturally-occurring peptide is a peptide which includes one or more non-naturally-occurring amino acids. In a preferred embodiment, the analog of THC possesses the therapeutically effective characteristics of THC described herein while lacking its psycho-active effects.

As used herein, “nicotinic receptor” is nicotinic acetylcholine receptors. The receptor is a ligand-gated ion channel receptor that interacts with acetylcholine and nicotine. The receptor is located in the membrane and on the surface of certain neurons and lymphoid cells. The nicotinic receptor is composed of five subunits arranged symmetrically to for pentamers around a central pore.

A “nicotinic compound” is an alkyloid and an “analog” of nicotine that possesses adequate homology to nicotine to function biologically as nicotine, but also possesses chemical structures which differ from nicotine. The nicotinic compound is a ligand of the nicotinic receptor. The analog may be naturally occurring or synthetic. An example of a synthetic analog of a naturally-occurring peptide is a peptide which includes one or more non-naturally-occurring amino acids.

The “therapeutically effective amount” for purposes herein is thus determined by such considerations as are known in the art. A therapeutically effective amount of the compounds of cannabinoid compounds and nicotinic compounds or any combination thereof with or without additional compounds is that amount necessary to provide a therapeutically effective result in vivo. The amount of cannabinoid compounds and nicotinic compounds or any combination thereof with or without additional compounds must be effective to achieve a response, including but not limited to total prevention of (e.g., protection against) and to improved survival rate or more rapid recovery, or improvement or elimination of symptoms associated with immune diseases, including without limitation Alzheimer's disease, autoimmune disorder, Parkinson's disease, multiple sclerosis, Tay Sach's disease, Rett Syndrome, lysosomal storage diseases, HIV dementia, prion disease, ischemia, ataxia, and amyotrophic lateral sclerosis, and other indicators as are selected as appropriate measures by those skilled in the art. In accordance with the present invention, a suitable single dose size is a dose that is capable of preventing or alleviating (reducing or eliminating) a symptom in a patient when administered one or more times over a suitable time period. The “therapeutically effective amount” of a compound of the present invention will depend on the route of administration, type of patient being treated, and the physical characteristics of the patient. These factors and their relationship to dose are well known to one of skill in the medicinal art.

“Administration” or “administering” is used to describe the process in which compounds of the present invention, alone or in combination with other compounds, are delivered to a patient. The composition may be administered in various ways including oral, parenteral (referring to intravenous and intraarterial and other appropriate parenteral routes), intrathecally, intramuscularly, subcutaneously, colonically, rectally, and nasally, transcutaneously, among others. Each of these conditions may be readily treated using other administration routes of compounds of the present invention to treat a disease or condition. The dosing of compounds and compositions of the present invention to obtain a therapeutic or prophylactic effect is determined by the circumstances of the patient, as known in the art. The dosing of a patient herein may be accomplished through individual or unit doses of the compounds or compositions herein or by a combined or prepackaged or pre-formulated dose of a compounds or compositions.

The pharmaceutical compositions of the subject invention can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjutants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W [1995] Easton Pa., Mack Publishing Company, 19^{th} ed.) describes formulations that can be used in connection with the subject invention.

A pharmaceutical composition of the invention is formulated to be compatible with its intended route of administration. Examples of routes of administration include parenteral, e.g., intravenous, intradermal, subcutaneous, oral, inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Both nicotine and Δ⁹-tetrahydrocannabinol (THC) are known to possess immune suppressing properties, but no previous studies have investigated their combined effects on innate immune system function, despite the fact that most marijuana users use both drugs together. While potentially detrimental during development, the ability of both nicotine and THC to suppress immune function may be therapeutic in treating chronic pro-inflammatory diseases. Upon activation, brain immune cells, known as “microglia,” release pro-inflammatory cytokines, such as TNF-α, which have been implicated in causing neuronal cell death.

Pairs of C57BL/6 mice were obtained from Jackson Laboratory (Bar Harbor, Me.). Murine primary culture microglial cells were isolated from newborn mouse cerebral cortices under sterile conditions and kept at 4° C. prior to mechanical dissociation. Cells were plated in 75 cm² flasks in RPMI medium supplemented with 5% fetal calf serum, 2 mM glutamine, 100 units/mL penicillin, 0.1 μg/mL streptomycin, and 0.05 mM 2-mercaptoethanol and kept for 14 days so only glial cells remain. The microglial cells were isolated by shaking the flasks at 200 rpm. More than 98% of the glial cells remaining stain positive for membrane attack complex-1 (CD-11b, Roche Diagnostics Corp., Indianapolis, Ind.). Additionally, between 85% and 95% of microglial cells usually stain positive for CD45 by fluorescence activated cell sorter (FACS) analysis.

On the day of sacrifice, mice were overdosed with pentobarbital (100 mg/kg). Blood was collected from the descending aorta, the aorta clamped and the heart perfused with saline. The brain was removed, bisected sagittally and each half separately immersed in freshly prepared 4% paraformaldehyde in 100 mM KPO₄ buffer (pH 7.4). The brain was post-fixed in paraformaldehyde for 24 hrs, one hemisphere will then be cryoprotected in a series of sucrose solutions, frozen, sectioned in the horizontal plane at 25 μm using a sliding microtome and stored at 4° C. in Dulbecco's phosphate buffered saline for immunocytochemistry and histology. All sections were collected to permit unbiased sampling of every 12th section throughout the brain with the quantitative histological procedures. Immunocytochemistry was performed on floating sections. Sections were incubated with the primary antibody overnight at 4° C., then incubated in biotinylated secondary antibody (2 hrs) followed by streptavidin-peroxidase. Peroxidase reactions consisted of 1.4 mM diaminobenzidine with 0.03% hydrogen peroxide in PBS for exactly 5 min. Nonspecific reaction product formation was negligible as assessed by omitting the primary antibody and/or preincubating antisera with appropriate antigen. Each assay was balanced with respect to the experimental groups.

Mouse brains were isolated under sterile conditions on ice and placed in ice-cold lysis buffer (20 mM Tris, pH7.5, 150 mM NaCl, 1 mM EDTA, 1 mM EGTA, 1% v/v Triton X-100, 2.5 mM sodium pyrophosphate, 1 mM 3-glycerolphosphate, 1 mM Na3VO4, 1 μg/mL leupeptin) with 1 mM PMSF. Brain tissues was then sonicated on ice for approximately 3 min, cooled on ice for 15 min, and then centrifuged at 15,000 rpm for 15 min. Total Aβ species were detected by acid extraction of brain homogenates in 5 M guanidine buffer or by 1% Triton X-100 extraction, followed by a 1:10 dilution in lysis buffer with 1 mM PMSF. Aβ₁₋₄₀, Aβ₁₋₄₂ and total Aβ levels (estimated by summing Aβ₁₋₄₀ and Aβ₁₋₄₂ values) were quantified in these samples using Aβ₁₋₄₀ and Aβ₁₋₄₂ ELISA kits (BioSource International, Invitrogen, Carlsbad, Calif.) in accordance with the manufacturer's instruction, except that standards include 0.5 M guanidine buffer. Total protein was quantified in brain homogenates using the Bio-Rad protein assay (Bio-Rad Laboratories, Inc., Hercules, Calif.); thus ELISA values were reported as ng of Aβ1-x/wetg of brain. Mouse EDTA-plasma was used neat at a 1:4 dilution in lysis buffer with 1 mM PMSF using the method described above for determination of plasma Aβ levels, and values was reported as pg/mL of Aβ.

sAPP-α ELISA was performed as previously described by Olsson (Olsson, A., et al., Measurement of alpha- and beta-secretase cleaved amyloid precursor protein in cerebrospinal fluid from Alzheimer patients. Exp. Neurol. 2003 September; 183(1):74-80) with minor changes. Briefly, high binding 96-well plates (Nunc, Denmark) were coated with monoclonal anti-Aβ₁₋₁₇ antibody (6E10) diluted in 100 μL (1 μg/mL) of carbonate buffer (pH 9.6) and incubated for overnight at 4° C. The plate was washed five times with PBS-Tween buffer (0.05% Tween 20) and blocked with 200 μL of blocking buffer (1% BSA in PBS) for 2 hrs at 37° C. All samples were analyzed in duplicate. Samples of cell cultured media, plasma and brain homogenates were diluted 1:1, 1:4 and 1:10 respectively in Reagent Diluent (1% BSA in PBS) and added to each well of the plate. The plate was incubated for 2 hrs at 37° C. After washing 5 times, 100 μL of goat anti-human N-terminal APP antibody (BioSource International, Inc., Camarillo, Calif.; diluted 1:3,000 in Reagent Diluent) was added to each well of the plates. Following 2 hour-incubation at 37° C. and 5-time washing, 100 μL of anti-goat IgG conjugated with HRP (1:1,500) was added to each well of the plates. The plate was incubated for 1 hr at 37° C. Following washing 5 times, 100 μL of substrate solution (TMB) was added to each well of the plate. 20 mm at room temperature later, 50 μL of stop solution (2 NH₂SO₄) was added to each well of the plate. The optical density was determined immediately by a microplate reader at 450 nm. Data was reported as ng/mL of sAPP-α in cell cultured media and plasma, or as ng of sAPP-α/wet g of brain homogenates.

During mouse brain lysis, an aliquot corresponding to 50 μg of total protein was electrophoretically separated using 16.5% Tris-tricine gels. Electrophoreses proteins were transferred to PVDF membranes (Bio-Rad), washed in dH₂O, and blocked for 1 hr at ambient temperature in Tris-buffered saline (TBS; Bio-Rad, Hercules, Calif.) containing 5% (w/v) non-fat dry milk. After blocking, membranes were hybridized for 1 hr at ambient temperature with various primary antibodies. Membranes were then washed 3 times for 5 min each in ddH₂O and incubated for 1 hr at ambient temperature with the appropriate HRP-conjugated secondary antibody (1:1,000, Pierce Biotechnology, Inc. Rockford, Ill.). All antibodies were diluted in TBS containing 5% (w/v) of non-fat dry milk. Blots was developed using the luminol reagent (Pierce Biotechnology, Thermo Fisher Scientific, Inc., Rockford, Ill.). Densitometric analysis was done using the Fluor-S MultiImager™ with Quantity One™ software (Bio-Rad Laboratories, Inc., Hercules, Calif.). Immunoprecipitation was performed for detection of sAPP-α, sAPP-β and Aβ by incubating 200 μg of total protein of each sample with various sequential combinations of 6E1 0 (1:100; Signet Laboratories, Dedham, Mass.) and/or 22C11 (1:100; Roche, Basel, Switzerland) overnight with gentle rocking at 4° C., and 10 μL of 50% protein A-Sepharose beads was then added to the sample (1:10; Sigma-Aldrich, Inc., St. Louis, Mo.) prior to gentle rocking for an additional 4 hrs at 4° C. Following a wash with cell lysis buffer, samples were subjected to Western blot as described above. Antibodies used for were APP carboxyl-terminal antibody 369 (1:1,000), anti-carboxyl-terminal APP antibody (1:500; Calbiochem, EMD Chemicals, Inc., Gibbstown, N.J.), anti-amino-terminal APP antibody 22C11, anti-amino-terminal Aβ antibodies BAM-10 (1:1,000; Sigma-Aldrich, Inc., St. Louis, Mo.) or 6E10 (1:1,000; Signet Laboratories, Dedham, Mass.), anti-ADAM-10 (Calbiochem), anti-TACE (Calbiochem) or anti-actin antibody (1:1,500; as an internal reference control; Roche), anti-phospho-tau antibodies (including ATB, PHF1 and AT270). A-, β-, γ-secretase activities was quantified in cell lysates and mouse brain homogenates using available kits based on secretase-specific peptides conjugated to fluorogenic reporter molecules (R&D Systems, Minneapolis, Minn.).

The primary cultured cells were plated in 24 well culture tissue plates at 5×10⁴ cells/well and pretreated at four time points (0.5, 1, 12, 24 hours) with either nicotine (0.1-10 μM), THC (0.1-10 μM), or combination of the two chemicals, and challenged with IFN-γ and Aβ (100 ng/mL). In addition, cannabinoid antagonists AM 251 (CB1) and AM 281 (CB2) were used (0.1-10 μM), cannabinoid agonists ACEA (CB1) and JHW 015 (CB2) were used (0.1-10 μM) in challenge experiments to determine receptor specificity. Experiments were conducted in triplicate and date combined for analysis.

Cell cultured media was collected for measurement of cytokines by commercial cytokine ELISA kits, as described previously. In parallel, cell lysates were prepared for measurement of total cellular protein. Data was represented as ng/mg total cellular protein for each cytokine production. Mouse brain homogenates from the hippocampus and anterior cortex was prepared and be used at a dilution of 1:10 in PBS for this assay. Brain tissue-solublized cytokines was quantified using commercially available ELISA kits (BioSource International, Inc., Camarillo, Calif.) that allow for detection of IL-1β, IL-6, IL-12p70 and TNF-α. Cytokine detection was carried out according to the manufacturer's instruction. The Bio-Rad protein assay was used to allow for normalization of values to total protein. Data was represented as ng/mg total cellular protein for each cytokine.

Because the 3 month treatment period is difficult to accomplish by either injection or oral gavage, 90 day drug delivery pellets was custom made and used for this project (Innovative Research of America, Saratoga, Fla.) to deliver nicotine alone (0.2 mg/kg/day), THC alone (0.3-3 mg/kg/day), or the combination as described for treatment groups in The Table. Doses were determined for a narrow dose range (0.3-3 mg/kg/day), and focused around the most likely therapeutic dose of 1 mg/kg per day.

The Table: Treatment Groups

| | Age @ start of | Nicotine | | Treatment |
|---|---|---|---|---|
| Mouse Type | treatment | (mg/kg) | THC (mg · kg) | duration |
| PSAPP | 8 months | 0 | 0 | 3 months |
| PSAPP | 8 months | 0.2 | 0.3 | 3 months |
| PSAPP | 8 months | 0.2 | 1.0 | 3 months |
| PSAPP | 8 months | 0.2 | 3.0 | 3 months |
| PSAPP | 8 months | 0.2 | 0 | 3 months |
| PSAPP | 8 months | 0 | 0.3 | 3 months |
| PSAPP | 8 months | 0 | 1.0 | 3 months |
| PSAPP | 8 months | 0 | 3.0 | 3 months |
| Wild Type | 8 months | 0 | 0 | 3 months |
| Wild Type | 8 months | 0.2 | 0.3 | 3 months |
| Wild Type | 8 months | 0.2 | 1.0 | 3 months |
| Wild Type | 8 months | 0.2 | 3.0 | 3 months |
| Wild Type | 8 months | 0.2 | 0 | 3 months |
| Wild Type | 8 months | 0 | 0.3 | 3 months |
| Wild Type | 8 months | 0 | 1.0 | 3 months |
| Wild Type | 8 months | 0 | 3.0 | 3 months |

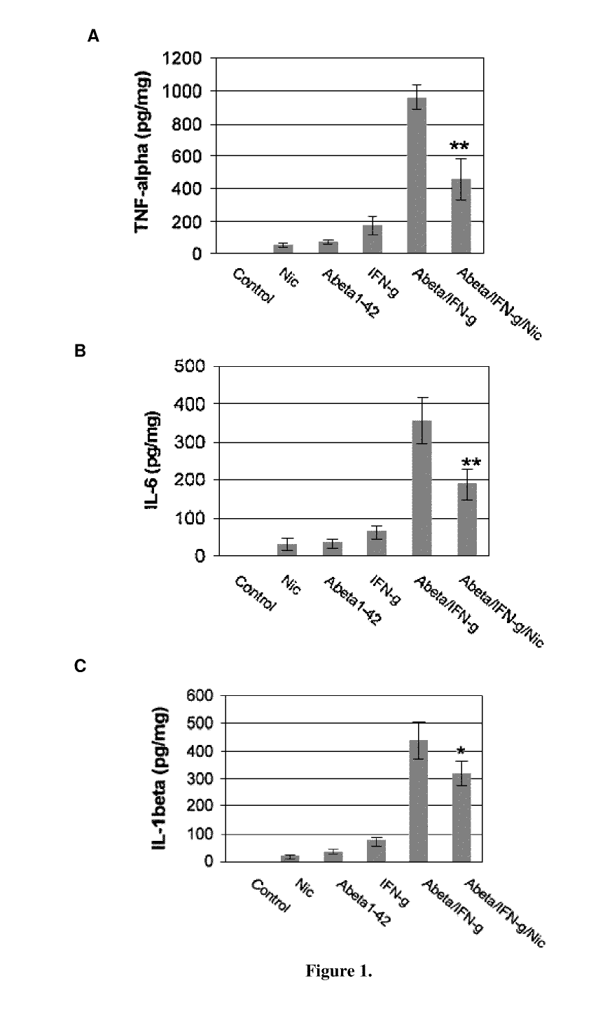

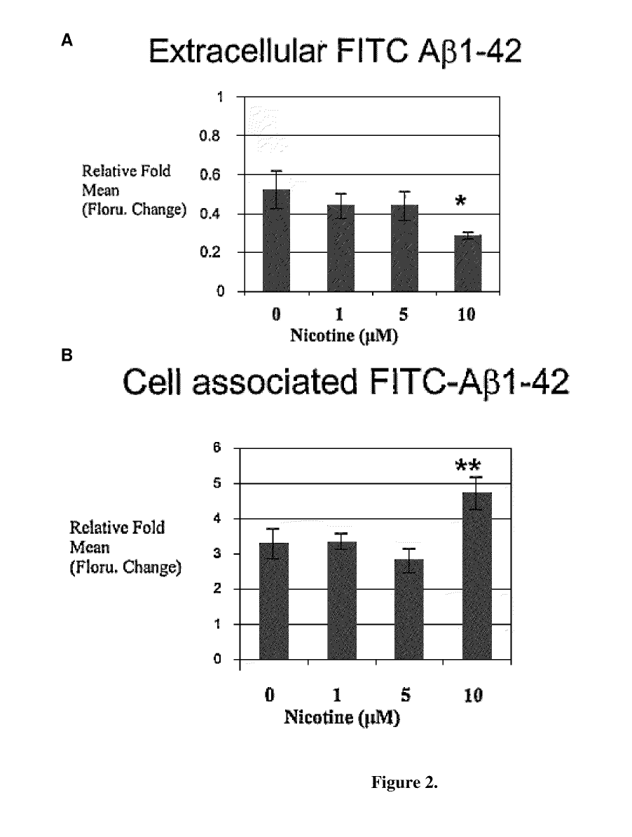

Immune reactions in Alzheimer's disease and prion-related encephalopathies (PRE) are dominated by microglia activation, with IL-6 released by reactive microglia a dominant cause of neuronal injury (Garção, P., Olivera, C. R., Agostinho, P., Comparative study of microglia activation induced by amyloid-beta and prion peptides: role in neurodegeneration. J. Neurosci. Res., 2006 July; 84(1):182-93). Functional nicotinic acetylcholine receptors (nAChR) have been observed on microglia. Moreover, administration of nicotine suppresses microglial activation produced by TNF-α and Aβ peptide challenge and enhances microglial phagocytosis (cellular uptake) of Aβ₁₋₄₂ peptide. Microglia cultures were exposed to stimulatory compounds, Aβ₁₋₄₂, IFN or both, and cytokine levels were measured. Administration of nicotine was found to effectively suppress microglial-released cytokines TNF-α, IL-6, and IL-1β, seen in FIGS. 1(A) through 1(C). It was also found that administration of nicotine enhanced microglial phagocytosis of Aβ₁₋₄₂ peptide, as evidenced by decreased extracellular and increased intracellular Aβ₁₋₄₂ protein seen in FIGS. 2(A) and 2(B).

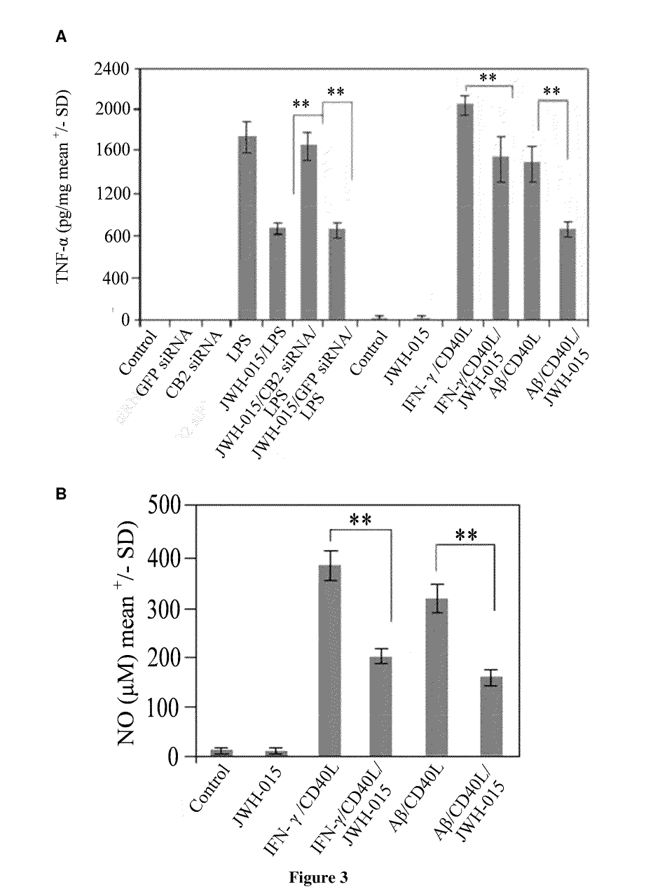

Cannabinoid receptors are expressed on microglia and regulate microglial function. As such, cannabinoid regulation of CD40 activation of microglial was investigated. N9 cells, transfected for 18 hr with specific murine CB2 targeting siRNA (100 nM), were treated for 4 hr with lipopolysaccharid (LPS), a positive control of microglia activation. The cells were then administered JWH-015 and TNF-α release was measured by ELISA, as seen in FIG. 3(A). Anti-CB2 siRNA was able to completely abolish JWH-015-mediated reductions in LPS-induced TNF-α release. Moreover, JWH-015 significantly reduced IFN-γ/CD-40L-induced NO production and Aβ/CD-40L-induced NO production, seen in FIG. 3(B).

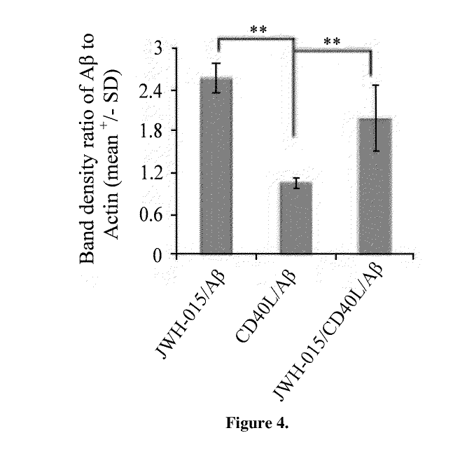

To examine the functional consequences of CB2 agonist treatment on CD40 expression, mouse primary microglial cells were stimulated with either IFN-γ/CD40L protein (Polazzi, E., A. Contestabile, Reciprocal interactions between microglia and neurons: from survival to neuropathology. Rev. Neurosci., 2002. 13(3): 221-242; Facchinetti, F., et al., Cannabinoids ablate release of TNFalpha in rat microglial cells stimulated with lypopolysaccharide. Glia, 2003. 41(2): 161-168; Walter, L., et al., Nonpsychotropic cannabinoid receptors regulate microglial cell migration. J. Neurosci. 2003. 23(4):1398-405) or Aβ₁₋₄₂/CD40L protein in the presence or absence of JWH-015 for 24 hr. ELISA measurements revealed that either IFN-γ/CD40L or Aβ₁₋₄₂/CD40L increased the secretion of the pro-inflammatory molecule TNF-α, as indicated in FIGS. 3(A) and (B). However, when CB2 is stimulated by the presence of JWH-015, these pro-inflammatory molecules were significantly reduced. The canonical microglial function in the CNS is thought to be phagocytosis, given that IFN-γ and CD40 signaling are maturation agents that oppose this phagocytic function. Murine primary microglial cultures were exposed to 3 μM of Aβ₁₋₄₂ (for immunoblotting) or Cy3™-Aβ₁₋₄₂ (for phagocytosis assay) in the presence or absence of CD40L protein or CD40L protein/JWH-015. After 3 hr, the amount of phagocytosed Aβ₁₋₄₂ peptide was determined by quantitative immunoblotting experiments, seen in FIG. 4. Aβ band densities were compared to β-actin, showing JWH-015 significantly reduced Aβ levels. CD40 ligation decreased microglial phagocytosis compared to controls, while CB2 agonist treatment alone increased phagocytosis compared to control. The presence of JWH-015 rescued microglial phagocytosis of Cy3-Aβ₁₋₄₂ following CD40L treatment. In a parallel experiment, CB2 stimulation by JWH-015 resulted in a significant attenuation of CD40L-mediated impairment of microglial phagocytosis of Aβ₁₋₄₂, as evidenced by increased band density ratio of Aβ to β-actin using Western immunoblotting. These data indicate that JWH-015 is activating CB₂ to oppose the TNF-a release caused by LPS treatment.

### Example 1

### Nicotinic/Cannabinoid Combination Treatment Mediates Suppression of Inflammation In Vitro

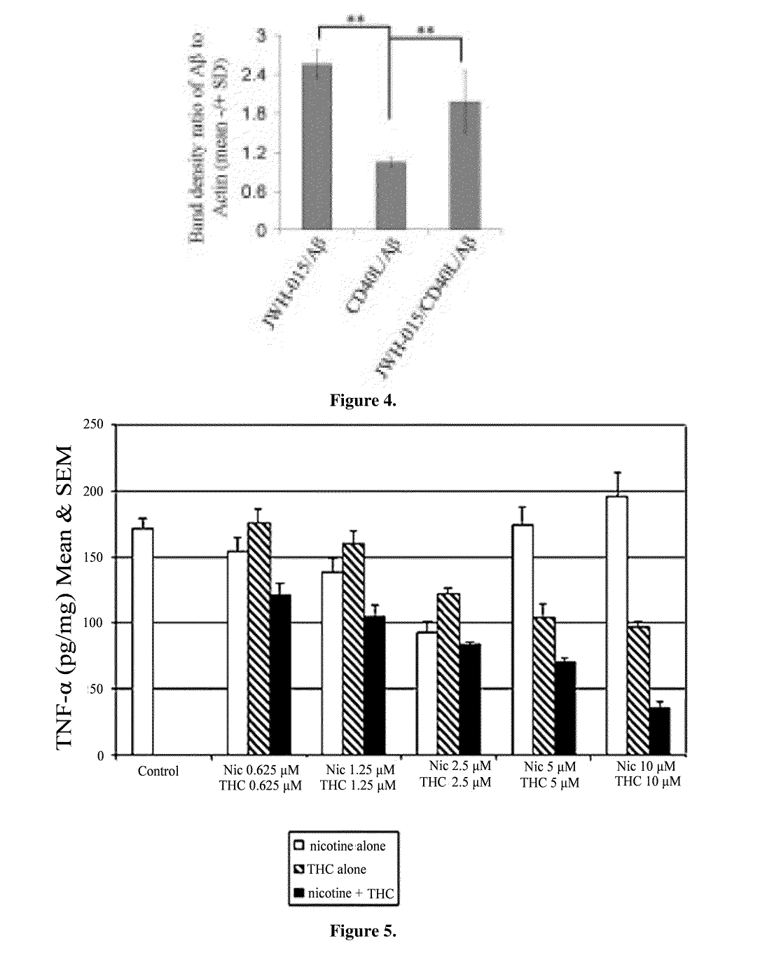

Cell cultures were treated with nicotinic/THC treatment, as described above. The concentration response and time-course functions of nicotine/THC treatment were analyzed against the cytokine profiles of microglial cells for TNF-α, IL-1β and IL-6, IL-12 induced by TNF-α and Aβ exposure. Each cytokine was represented as pg of cytokine/mg of total cellular protein. Data was analyzed using ANOVA with post hoc comparison using Bonferroni's or Dunnett's T3 methods as determined by Levene's test for equality of the variances. The combination nicotinic/THC treatment is dose dependent and has synergistic effects in attenuating microglia activation, as seen on TNF-α analysis in FIG. 5.

These findings suggest that the combination of THC and nicotine clinically have greater efficacy in reducing neuroinflammation with less side effects than either drug given alone. Because of nicotine's short half-life and side effects from oral administration, a transdermal formulation or an oral spray formulation (considering similar THC formulations in patent literature) comprised of both THC and nicotine would appear to be the most effective therapeutic approach to treating any central nervous system disorder involving microglial activation. This is also relevant to peripheral inflammation thru macrophage activation. In addition, other cannabinoids and other nicotinic-like medications currently in development are also envisioned for this treatment.

### Example 2

### Nicotinic/Cannabinoid Treatment Effects of Immune Phagocytosis

Nicotinic and cannabinoid compounds have dose-dependent synergistic effects in attenuating microglia activation. Concentration-response and time-course functions for microglial phagocytosis (cellular uptake) of Aβ₁₋₄₂ peptide were then characterized. Treatment with nicotine (10 μM) or THC (5 μM) markedly decreased extracellular FITC-Aβ₁₋₄₂ remaining in the supernatant while increasing cell-associated FITC-Aβ₁₋₄₂, as seen in FIGS. 2(A) and (B) and 4. This indicates increased capacity of microglial phagocytosis. To fully characterize these effects, primary microglial cells cultured in 24-well tissue-culture plates (1×10⁵/well) were treated for 0, 15, 30, 60, 120, and 180 mm with “aged” FITC tagged Aβ₁₋₄₂ peptide at 300 nM in the presence or absence of nicotine (0.1-10 μM), THC (0.01-10 μM), and their combination. The microglia were then administered nicotine (0.1-10 μM), THC (0.01-10 μM) and a combination. In addition, cannabinoid antagonists, AM 251 (CB1) and AM 281 (CB2) (0.01-10 μM), cannabinoid agonists, ACEA (CB1) and JHW 015 (CB2) (0.01-10 μM), and nicotinic receptor antagonists, mecamylamine (α4β2) and α-bungorotoxin (α 7) (0.01-10 μM) were used in challenge experiments to determine receptor specificity. For fluorescence analysis, the cells were then washed 5 times with ice-cold PBS to remove the extracellular Aβ, and fixed in 4% paraformaldehyde. The cells were mounted and viewed under an Olympus IX71/IX51 fluorscence microscope equipped with a digital camera system. Image Pro software was used to quantify fluorescence signals, using a minimum of 5 random fields. For fluorometric analysis, microglial cells were treated in parallel, rinsing the cells 3 times with medium and the cells lysed. Extracellular and cell associated FITC tagged Aβ was quantified using an MFX 96-well microplate fluorometer (Molecular Devices, MDS, Inc., Sunnyvale, Calif.) with an emission wavelength of 538 nm and an excitation wavelength of 485 nm. A standard curve from 0 nM to 500 nM of FITC-tagged Aβ was run for each plate. The total cellular protein of all groups was quantified using the Bio-Rad protein assay. In addition, in parallel 24-well tissue-culture plates, microglial cells was incubated at 4° C. with FITC-conjugated Aβ with or without various combinations of CD40L as controls for non-specifically incorporated Aβ. Microglial cells was then rinsed 3 times in Aβ-free complete medium, and the media was exchanged with fresh Aβ-free complete medium for 10 min both to allow for removal of non-incorporated Aβ and to promote concentration of the Aβ into phagosomes. The mean fluorescence values for each sample at 37° C. and 4° C. at the indicated time points were determined by fluorometic analysis. Relative fold change values were calculated as: (mean fluorescence value for each sample at 37° C./mean fluorescence value for each sample at 4° C.). In this manner, both extracellular and cell associated FITC-labeled Aβ were quantified. Combination treatment with nicotine and THC caused microglial phagocytosis of Aβ (data not shown). Further, the combination of nicotine and THC has a synergistic effect beyond the effects caused by THC or nicotine alone (data not shown).

For phagocytosis experiments, an important concern is the possibility of extracellular association of Aβ with microglial cell membrane. To address this issue, microglial cells was rinsed in Aβ-free complete medium, and media was exchanged with fresh Aβ-medium for 10 mm both to allow for removal of non-specifically incorporated Aβ and to promote concentration of the Aβ into phagosomes. Microglial cells were further cultured in parallel 24-well plates incubated at 4° C. with FITC-tagged Aβ at the same time points in the presence or absence of the appropriate treatment as control for non-specifically incorporated Aβ. Finally, immunoprecipitation and Western blot analysis of extracellular and cell associated Aβ by was performed using anti-Aβ antibodies (including anti-N-terminal-Aβ and anti-C-terminal Aβ antibodies). These tests confirmed the combination of nicotine/THC possess a synergistic effect on increasing microglial phagocytosis of Aβ that is not due to Aβ microglial membrane adherence (data not shown). Further, these effects were mediated by α7 nicotinic receptors and cannabinoid CB2 receptors for nicotine and THC, respectively.

### Example 3

### Nicotinic/Cannabinoid Treatment Mediates Suppression of Inflammation In Vivo

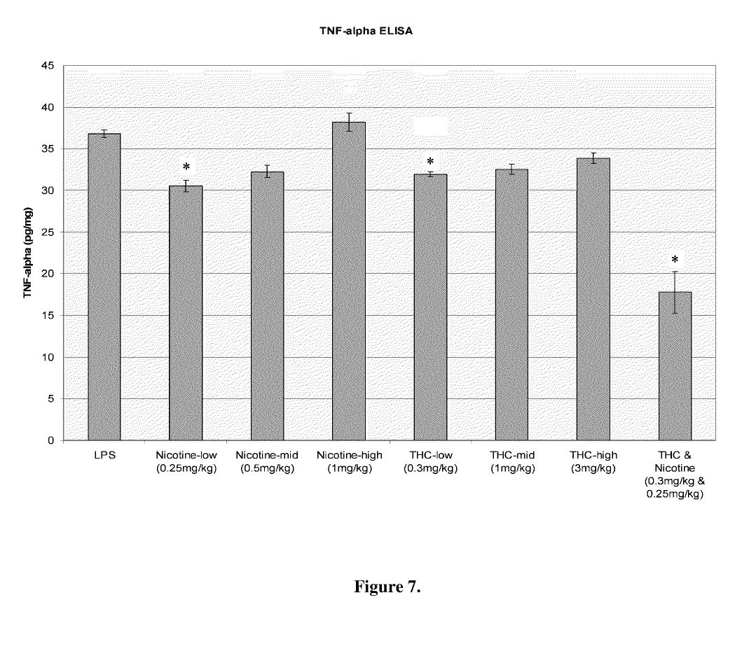

TNF-α levels of homogenized brains were examined using ELISA. Adult male C57/BLB mice received nicotine or THC on the right side of the abdomen as indicated. Treatment was immediately followed by 1 mg/kg LPS on the left side of the abdomen. Two hours later, mice were euthanized and brains were removed for TNF-α cytokine analysis. Bio-Rad protein assay was performed to measure total cellular protein. The combination of THC and nicotine reduce TNF-α levels in the mice, below levels observed in THC or nicotine only levels, seen in FIG. 7, indicating THC/nicotine treatment synergistically reduces microglial activation.

### Example 4

### Nicotinic/Cannabinoid Treatment Effects of Immune Phagocytosis In Vivo

Aβ deposition was measured by ELISA for Aβ₁₋₄₀/Aβ₁₋₄₂ and sAPP-α and Western blot for CTFs and sAPP-α/β. Starting at 6 weeks prior to euthanasia behavioral performance was assessed to determine if the treatments protect transgenic mice from developing cognitive impairments or if the treatments result in cognitive impairment in normal mice. Further, α, β, and γ-secretase cleavage activity was measured using fluorescence/ELISA kits (R&S systems) as described previously (Tan et at., 2002). To characterize microglia-associated inflammation, immunohistochemistry was performed for microglial markers CD36, CD80/86, CD40, MHC-II, p44/42 MAPK and p38 MAPK.

The reduction in β-amyloid plaque observed in these studies of nicotine treatment was comparable to that observed by Nordberg in 16 month APP mice receiving 8.5 months Aβ immunization. In Nordberg's study of short-term nicotine treatment, 9-month-old Tg2576 transgenic mice were injected subcutaneously (s.c.) twice daily for 10 days with either nicotine (0.45 mg/kg (free base) per day; Sigma-Aldrich Corp., St. Louis, Mo.) or saline. Because the magnitude of the reduction in insoluble Aβ₁₋₄₀ and Aβ₁₋₄₂ peptides after 10 days of nicotine treatment at this dose was so large (˜80%), half the dose of nicotine (0.2 mg/kg/day) was used to avoid having a ceiling effect and missing the potential interaction between nicotine and THC. Administration of nicotine/THC was observed to increase the generation of α-CTF and sAPP-α in the brain, while decreasing the levels of Aβ generation (data not shown). ELISA characterization of “total Aβ” primary antiserum (rabbit anti-Aβ₁₋₄₀, Paul Gottschall, USF) indicates the major epitope is within amino acids 1-16. Immunolabeling by this antiserum was blocked by preabsorption with either Aβ₁₋₄₀ or Aβ₁₋₄₂ peptide. Non-transgenic mice did not deposit Aβ, confirming the absence of nonspecific antibody reactivity.

To further validate the results, nicotine/THC treatment was administered to PSAPP mice (APP5w, PSENIdE9; Jankowsky, J. L., et al., Co-expression of multiple transgenes in mouse CNS: a comparison of strategies. Biomol. Eng. 2001. 17(6): 157-65) to confirm reduced AD-like pathology (including amyloidosis and microglia-associated inflammation) and reduced cognitive impairment in vivo. Double transgenic mouse strains expressing both a mutant human presenilin and amyloid precursor protein were used. Due to the double mutation, this APPsw/PSEN1dE9 transgenic strain (PSAPP) develops brain β-amyloid deposits by 8 months of age allowing for more expedient pharmacological testing.

Nicotine, THC, nicotine/THC, or control was administrated via drug delivery pellets as described above. The nicotine/THC was administered to PSAPP mice after the development of AD-like pathology (therapeutic treatment). For the therapeutic treatment, 128 eight-month-old PSAPP mice and 128 non-transgenic wild type littermates were included for comparison with the multiple transgenic treatment groups. At 11 months of age, following 3 months of therapeutic treatment with nicotine/THC, the PSAPP and non-transgenic (wild type) mice were sacrificed and blood withdrawn. Mice were perfused with saline and the brain bisected sagitally with the left half immersion fixed in paraformaldehyde for histological processing and the right half dissected into hippocampus, anterior cortex, posterior cortex, striatum, diencephalon, cerebellum and rest of brain. All dissections were rapidly frozen for subsequent biochemical analyses. Fluids from the blood and brain were measured for both nicotine and THC levels by an external laboratory (Quest Diagnostics, Inc., Tampa, Fla.).

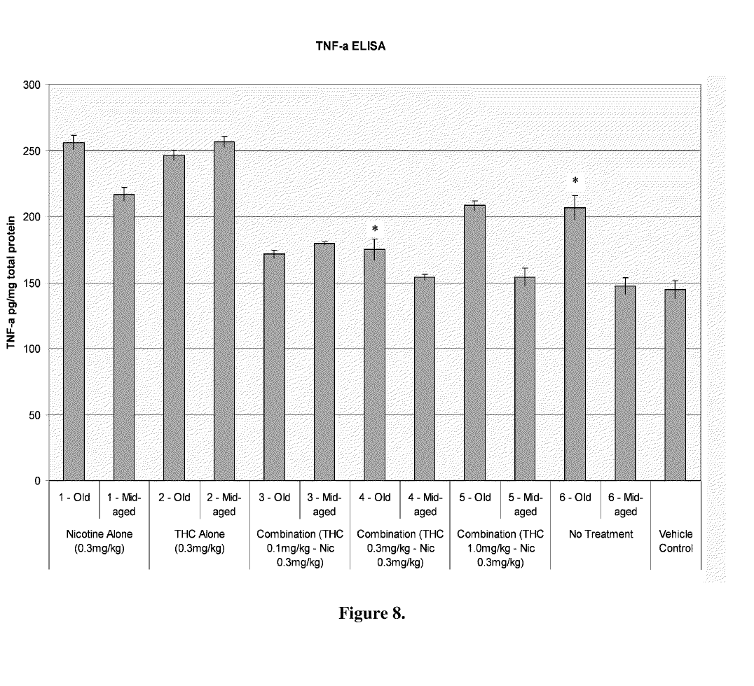

TNF-α levels were determined. Old PSAPP mice (16+ months of age) and mid-aged mice (11-12 months old) were i.p. injected once daily for two weeks with nicotine and THC, administered alone and in combination, as indicated. After the two week injection period, mice were sacrificed and tissues were collected for analysis of TNF-α levels. Treatment significantly reduced TNF-α in old animals receiving a combination dose and the old animal controls, seen in FIG. 8.

These findings, along with findings that nicotine and cannabinoid receptor activation, indicate cannabinoid/nicotinic compound treatment attenuates microglial activation and increases microglial phagocytosis of Aβ, chronic treatment with nicotine and THC attenuated AD-like pathology in Alzheimer transgenic mice. Nicotinic receptors are functionally expressed on microglia and nicotine reduced microglial activation and enhanced microglial phagocytosis of Aβ₁₋₄₂, a peptide implicated in AD. In addition, cannabinoid receptor activation similarly reduced microglial activation and enhanced microglial phagocytosis of Aβ₁₋₄₂ via inhibition of the CD40 signaling pathway. Moreover, the combination of nicotine and THC had dose-dependent and synergistic effects on reducing microglial activation. Therefore, the combination of nicotine and THC represents a powerful therapeutic strategy against pro-inflammatory diseases like AD.

In the preceding specification, all documents, acts, or information disclosed does not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

While there has been described and illustrated specific embodiments of a method of modulating inflammatory disease and treating a neurodegenerative disease, it will be apparent to those skilled in the art that variations and modifications are possible without deviating from the broad spirit and principle of the present invention. It is also to be understood that the following claims are intended to cover all of the generic and specific features of the invention herein described, and all statements of the scope of the invention which, as a matter of language, might be said to fall therebetween. Now that the invention has been described,

What is claimed is:

1. A method of modulating inflammatory response in a patient comprising the step of administering a therapeutically effective amount of a composition further comprising at least one cannabinoid and at least one nicotinic compound.

2. The method of claim 1, wherein the at least one cannabinoid is a cannabinoid-2 receptor agonist selected from the group consisting of delta-9-tetrahydrocannabinol, cannabidiol, dronabinol, JHW 015, anandamide, 2-arachidonyl glyceride, 2-arachidonyl glyceryl ether, O-arachidonoyl-ethanolamine, nabilone, PRS-211,092, CP 55,940 WIN-55212-2, JWH 133, SR 144528, and levonantradol.

3. The method of claim 1, wherein the at least one nicotinic compound is selected from the group consisting of nicotine, epibatidine, acetylcholine, cytosine, carbachol, dimethlphenylpiperazimium, and varenicline.

4. The method of claim 1, wherein inflammatory response is microglia-activated Th1 and Th2 immune responses.

5. The method of claim 1, wherein the therapeutically effective amount of the composition is administered intrathecally, subcutaneously or intravenously.

6. The method of claim 5, wherein the nicotinic compound is administered at 0.2 mg/kg/day and the cannabinoid is administered within the range of 0.3 and 3 mg/kg/day.

7. The method of claim 1 wherein the inflammatory response is induced by LPS.

8. A method of treating a neurodegenerative disease in a patient comprising the step of administering a therapeutically effective amount of a composition further comprising at least one cannabinoid and at least one nicotinic compound.

9. The method of claim 8, wherein the at least one cannabinoid is a cannabinoid-2 receptor agonist selected from the group consisting of delta-9-tetrahydrocannabinol, cannabidiol, dronabinol, JHW 015, anandamide, 2-arachidonyl glyceride, 2-arachidonyl glyceryl ether, O-arachidonoyl-ethanolamine, nabilone, PRS-211,092, CP 55,940 WIN-55212-2, JWH 133, SR 144528, and levonantradol.

10. The method of claim 8, wherein the at least one nicotinic compound is selected from the group consisting of nicotine, epibatidine, acetylcholine, cytosine, carbachol, dimethlphenylpiperazimium, and varenicline.

11. The method of claim 8, wherein the composition modulates microglia-activated Th1 and Th2 immune responses.

12. The method of claim 8, wherein the therapeutically effective amount of the composition is administered systemically.

13. The method of claim 12, wherein the therapeutically effective amount of the composition is administered intrathecally or intravenously.

14. The method of claim 8, wherein the nicotinic compound is administered at 0.2 mg/kg/day and the cannabinoid is administered within the range of 0.3 and 3 mg/kg/day.

15. The method of claim 8, wherein the neurodegenerative disease is selected from the group consisting of Parkinson's disease, Alzheimer's disease, multiple sclerosis, Tay Sach's disease, Rett Syndrome, lysosomal storage diseases, HIV dementia, prion disease, ischemia, ataxia, and amyotrophic lateral sclerosis.

16. The method of claim 15, wherein the neurodegenerative disorder is amyotrophic lateral sclerosis.

17. A composition comprising at least one cannabinoid and at least one nicotinic compound.

18. The composition of claim 17, wherein the at least one cannabinoid is a cannabinoid-2 receptor agonist selected from the group consisting of delta-9-tetrahydrocannabinol, cannabidiol, dronabinol, JHW 015, anandamide, 2-arachidonyl glyceride, 2-arachidonyl glyceryl ether, O-arachidonoyl-ethanolamine, nabilone, PRS-211,092, CP 55,940 WIN-55212-2, JWH 133, SR 144528, and levonantradol.

19. The composition of claim 17, wherein the at least one nicotinic compound is selected from the group consisting of nicotine, epibatidine, acetylcholine, cytosine, carbachol, dimethlphenylpiperazimium, and varenicline.

20. The composition of claim 17, wherein the composition is administered intrathecally, subcutaneously or intravenously.

21. The composition of claim 17, wherein the nicotinic compound is administered at 0.2 mg/kg/day and the cannabinoid is administered within the range of 0.3 and 3 mg/kg/day.
